(19) United States
(12) Patent Application Publication
(10) Pub. No.: US 2020/0377436 A1
(43) Pub. Date: Dec. 3, 2020

(54) OXIDATION OF LIMONENE

(71) Applicant: Symrise AG, Holzminden (DE)

(72) Inventors: Erich Dilk, Holzminden (DE); Detlef Geisel, Holzminden (DE); Stefan Lambrecht, Hehlen (DE)

(21) Appl. No.: 16/998,163

(22) Filed: Aug. 20, 2020

Related U.S. Application Data
(63) Continuation of application No. 15/575,076, filed on Nov. 17, 2017, now abandoned.

(30) Foreign Application Priority Data
May 21, 2015 (EP) 15168597.1

(51) Int. Cl. C07C 29/48; C07C 29/132; C07C 407/00

(52) U.S. Cl. CPC C07C 29/48; C07C 29/132; C07C 2601/14; C07C 2601/16; C07C 407/00

(57) ABSTRACT

The invention discloses a process for the oxidation of limonene, comprising the reaction of limonene with hydrogen peroxide in the presence of a catalyst containing atoms and/or ions of at least one metal, selected from the group consisting of molybdenum, tungsten, scandium, vanadium, titanium, lanthanum, zirconium, praseodymium, neodymium, samarium, europium, terbium, dysprosium, erbium or ytterbium, characterised in that the molecular weight of the catalyst is less than 2,000 g/mol and that the reaction is performed at a pH value of more than 7.5.

### FIELD OF THE INVENTION

The invention relates to a process for the production of oxidized derivatives of limonene, particularly of epoxy derivatives and peroxide derivatives, and particularly preferably of peroxide derivatives, and the use of these compositions for the production of perfumes, aromas or flavours, or the use of these compositions as intermediate products for the production of perfumes, aromas or flavours, respectively.

### STATE OF THE ART

The value of essential oils as perfumes, aromas or flavours is well known. The aromatic main constituents of citrus essential oils are monoterpenes, sesquiterpenes and the oxygen-containing derivatives thereof. The sensory properties of citrus aromas mainly depend on the content of oxygen-containing terpene derivatives, alcohols, aldehydes, esters and ketones.

It is also known that monoterpenes, particularly limonene, negatively influence the sensory properties of citrus aromas due to their high concentration. Therefore, various processes for the separation of limonene have been developed.

Limonene is a naturally occurring chemical compound which is classified as belonging to the group of terpenes.

For example, Fan et al discloses the separation of monoterpenes in J. Agric. Food Che., 2004, 52(16), 5162-5167, inter alia, of limonene, by combining the methods of conventional vacuum distillation with supercritical carbon dioxide extraction.

Another option is to convert limonene into valuable compounds which themselves are valuable perfumes, aromas or flavours, or into intermediate compounds which may be further processed to form part of the most diverse perfumes, aromas or flavours. For example, it is generally known that limonene is used as an educt for the production of particular perfumes, aromas or flavours, and also of intermediate compounds thereof.

In the course of our search for valuable perfumes, aromas or flavours, oxidized derivatives of limonene, particularly epoxy derivatives and peroxide derivatives, and particularly preferably peroxide derivatives proved to be particularly interesting compounds. For example, compounds 1-6 are mentioned:

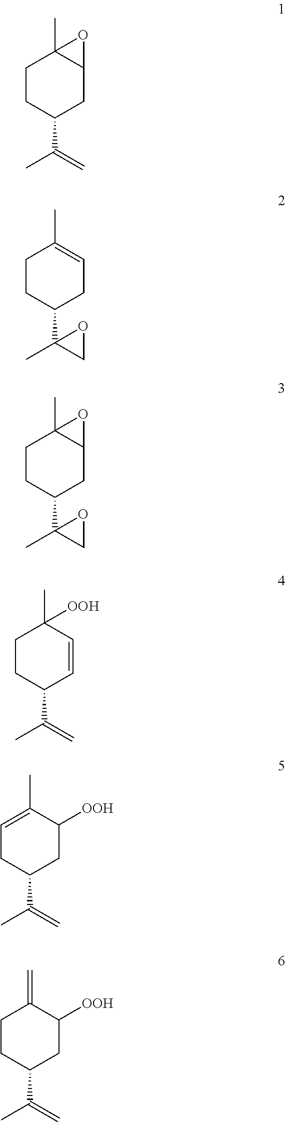

A known process for the production of peroxide derivatives of the type of compounds 4-6 comprises the photochemical oxidation of limonene in the presence of hydrogen peroxide (H₂O₂). The hydrogen peroxide mixture that was obtained as an intermediate in this process is transferred to the corresponding alcohol mixture by treating it with sodium sulfite solution (e.g., G. O. Schenk et al, Liebigs Ann. Chemie, 674 (1964), 93-117).

The disadvantage of photochemical production processes is that they are carried out in specific photo-reactors. In comparison with typical chemical reactors, this specific equipment requires higher investments and is, therefore, less common. In addition, the operation of photo-reactors requires much effort.

J. Am. Chem. Soc., 1968, 90, 975 describes a singlet oxygen oxidation (¹O₂-Ox), in which ¹O₂ is not photochemically generated, but chemically. In doing so, hydrophobic substrates are oxidized in a solvent mixture consisting of water and an organic solvent by means of a hypochlorite/H₂O₂ system. However, this process has found merely a few synthetic applications, as many substrates are not easily soluble in the required medium. In addition, applications are quite restricted as a result of side reactions between hypochlorite and the substrate or the solvent. Apart from that, a large part of the ¹O₂ is deactivated in the gaseous phase. Further, this process is not suitable for production on an industrial scale, as hypochlorite is added to H₂O₂ in the organic medium, and a large excess of H₂O₂ is required to suppress the side reaction of the substrate with hypochlorite. A further disadvantage is caused by the occurrence of stoichiometric amounts of salt.

Document WO 2009/033247 A2 describes the oxidation of an essential oil of laranja pêra (Citrus sinensis) which contains 96% limonene, with a 35% H₂O₂ solution. In this process, a heterogeneous catalyst of the general formula L/MₓN_{y}, where M=Zr, Al, Si, Ti; N═O; x and y=2 or 3, and L=Co, Ti, V, Cr, Mn, Fe, Cu, Mo, W, Re is used. According to the process described, the reaction mixture is heated for 24 hours at a temperature of 25° C. to 125° C. Under these conditions, 40-65% of the limonene was converted to oxygenated products such as Carvone, Carveol and limonene epoxides. The document does not indicate the specific catalyst used. A disadvantage of this process is the duration of the oxidation reaction, which is 24 hours. As a result, it is difficult to control the selectivity of the reaction in the direction of the formation of epoxy derivatives and peroxide derivatives, particularly, the formation of peroxide derivatives. In fact, document WO 2009/033247 A2 does not disclose the formation of peroxide derivatives of limonene.

Document U.S. Pat. No. 3,014,047 A discloses the oxidation of d-limonene to form peroxide derivative by means of air within a range of temperature of 25-80° C. Again, the duration of the oxidation reaction is a disadvantage. In this case, the duration of the oxidation reaction is from about 5 hours at 80° C. to more than six days at 25° C. Better results were obtained when the reaction was performed at 80° C. for 30 hours. Attempts to improve the results by increasing the reaction time were not successful, as the peroxide derivatives formed tend to disintegrate under these conditions.

Therefore, there is a strong demand for new processes for the production of oxidized derivatives of limonene, particularly of epoxy derivatives and peroxide derivatives, and particularly preferably of peroxide derivatives, which require shorter reaction times and are performed under conditions which reduce the disintegration of the compounds obtained.

The task of the present invention was, therefore, to provide an industrially employable process for the production of oxidized derivatives of limonene, particularly of epoxy derivatives and peroxide derivatives, and particularly preferably of peroxide derivatives, which improves the disadvantages of the state of the art described above. In doing so, the produced compounds should have advantageous sensory (with respect to aroma and flavour) properties, and the reaction should be carried out fast, economically, and on an industrial scale.

A second task of the present invention was, therefore, to provide an industrially applicable process for the production of oxidized derivatives of limonene, particularly of epoxy derivatives and peroxide derivatives, and particularly preferably of peroxide derivatives, which may be further processed to form part of various perfumes, aromas or flavours in a simple manner.

### DESCRIPTION OF THE INVENTION

The subject matter of the present invention is a process for the oxidation of limonene, comprising the reaction of limonene with hydrogen peroxide in the presence of a catalyst containing atoms and/or ions of at least one metal, selected from the group consisting of molybdenum, tungsten, scandium, vanadium, titanium, lanthanum, zirconium, praseodymium, neodymium, samarium, europium, terbium, dysprosium, erbium or ytterbium, characterised in that the molecular weight of the catalyst is less than 2,000 g/mol and that the reaction is performed at a pH value of more than 7.5.

Surprisingly, it was found that, by means of the above mentioned processes, oxidized derivatives of limonene, particularly epoxy derivatives and peroxide derivatives, and particularly preferably peroxide derivatives, may be produced such that the oxidation reaction is performed in reaction times that are significantly shorter than the reaction times described in the state of the art.

Beyond that, it was surprisingly found that performing the reaction at a pH value of more than 7.5 increases the stability of the products obtained and reduces the formation of undesired side products at the same time.

In a preferred embodiment, the molecular weight of the catalyst is less than 1,000 g/mol, and particularly preferably it is less than 500 g/mol.

The metals can also be present in the form of, for example, oxo complexes, oxides, hydroxides, salts such as, for example, nitrates, carboxylates, carbonates, chlorides, fluorides, sulfates or tetrafluoroborates.

In particular, the catalysts can comprise atoms and/or ions of molybdenum, tungsten, scandium, vanadium, titanium and lanthanum, and particularly preferably of molybdenum and tungsten.

In particular, the catalysts can consist of or contain the following compounds: sodium molybdate, sodium molybdate dihydrate, sodium tungstate, sodium tungstate dihydrate and lanthanum nitrate. The catalysts can be used, particularly, in solid or in dissolved form.

In particular, the reaction can occur in at least one organic solvent. For example, the solvents can be selected from the group consisting of C1 to C8 alcohols and amides. The solvent can also be selected from the group consisting of methanol, ethanol, propanol, isopropanol, ethylene glycol, propylene glycol, N-methylformamide, dimethylformamide or N-methyl pyrrolidone. Optionally, the at least one solvent can contain up to 30% by weight water.

It appeared that the oxidation reaction is carried out, particularly preferably, at a pH value of more than 8, and more preferably of more than 9. Therefore, in a preferred embodiment, the reaction of limonene with hydrogen peroxide in the presence of the catalyst is carried out at a pH value of more than 8, and more preferably of more than 9. Under these conditions the formation of degradation products of limonene is reduced particularly favourably, especially degradation products that are formed by rearrangements, cleavage of the double bonds and dehydrations.

It also appeared that the oxidation reaction is carried out particularly preferably at temperatures between 25 and 90° C., more preferably between 40 and 90° C.

In a further preferred embodiment, the oxidation reaction is carried out at a pH value of more than 8 and at temperatures within the range of 40 and 90° C., particularly preferably at a pH value of 9 and at temperatures within the range of 40 and 90° C.

Further advantageous results can be obtained with an amount of catalyst of 1 to 50 mole percent, based on limonene and/or 2-10 molar equivalents of hydrogen peroxide per 1 mole limonene.

The products formed are mainly hydroperoxides of limonene. Therefore, another subject matter of the invention is the use of the above-described process for the production of an aroma composition, comprising at least one peroxide derivative, preferably two or more peroxide derivatives, of limonene.

The mixture of oxidized derivatives obtained by the process according to the present application can be achieved by conventional separation methods, for example, by gas chromatography-mass spectrometry (GC-MS), by high-performance liquid chromatography (HPLC), or by fractional distillation.

Optionally, the mixture of oxidized derivatives obtained, particularly hydroperoxides of limonene, can be directly processed further, i.e. without an isolation step, to produce valuable perfumes, aromas or flavours.

Accordingly, in a further embodiment, the mixture of hydroperoxides of limonene obtained by the oxidation process according to the present application is directly reacted with a reducing agent.

Accordingly, another subject matter of the present invention is a process for the production of hydroxy derivatives of limonene, comprising the following steps:
(a) Oxidation of limonene and
(b) Reaction of the mixture obtained in step (a) with a reducing agent.

The preferred reducing agent is sodium sulfite.

For example, the reduction can be performed by introducing the reaction products of the oxidation into an aqueous sodium sulfite solution and subsequent stirring at a temperature of 25 to 90° C. and at a pH value of more than 7.5.

Subsequently, the reaction mixture obtained is separated by conventional separation methods such as, for example, fractional distillation. Based on the reaction mixture, for example, p-2,8-Menthadien-1-ol, p-1(7),8-Menthadien-2-ol and Carveol may be obtained, as is illustrated in the examples of the present application.

The reactions may be performed both by means of batch processing and continuous processing. A nitrogen flow may be passed through the reaction apparatus during the reactions.

In another possible but non-limiting example of a batch-wise reaction, limonene, methanol and the catalyst, optionally dissolved in water, are present, and hydrogen peroxide solution is added at the selected reaction temperature. After the end of the reaction, the obtained hydroperoxides are reduced by feeding them into a sodium sulfite solution.

In a possible but non-limiting example of a continuous process, the raw materials are fed into the lower portion of a tube reactor, and the reaction mixture which had finished reacting is either received in the upper portion or passed into the sodium sulfite solution.

### INDUSTRIAL APPLICATION

According to the present invention, oxidized derivatives of limonene can be produced, particularly of epoxy derivatives and peroxide derivatives, and particularly preferably of peroxide derivatives which themselves can be reacted to perfumes, aromas or flavours, or which can be further processed to form part of valuable perfumes, aromas or flavours.

### EXAMPLES

The present invention will be more easily understood with reference to the following examples. However, these examples are merely intended to illustrate the invention und cannot be interpreted as limiting with regard to the scope of protection of the invention.

### Example 1

2.72 g D-limonene in 24.4 g methanol are placed into a 100 ml three-necked flask apparatus with stirrer. 0.22 g sodium molybdate dihydrate, dissolved in 1.86 g water, is added and adjusted to pH=10 by means of 5% sodium hydroxide. Heat is applied until reaching the return temperature, and 4 g 50% hydrogen peroxide is fed in within 30 minutes and is allowed to continue reacting for another 5 minutes. Subsequently, the reaction mixture is added to a solution of 2.5 g sodium sulfite in 7.2 g water at 60° C. and stirred for 3 hours until a complete peroxide degradation is achieved. The product is filtered off the precipitated sediment, and the filtrate is concentrated in a rotation evaporator. The composition of the raw product according to GC area % is indicated in Table 1.

### Example 2

2.72 g D-limonene in 11 g methanol and 7 g water are placed into a 100 ml three-necked flask apparatus with stirrer. Further processing was performed analogous to example 1.

### Example 3

Performed analogous to example 2 but using 0.3 g sodium tungstate dihydrate, dissolved in 1.86 g water.

### Example 4

Performed analogous to example 1, but using 0.4 g lanthanum nitrate, dissolved in 1.86 g water.

Table 1 shows that the yield of the produced quantities of the compounds p-2,8-Menthadien-1-ol, p-1(7),8-Menthadien-2-ol and Carveol, in all, is very high. In addition, it is apparent that the ratio of the substances between themselves varies with the different processes of production.

### Example 5

27.2 g D-limonene in 244 g methanol are placed into a 1 L double jacketed tank with stirrer. 4.4 g sodium molybdate dihydrate, dissolved in 18.6 g water, is added and adjusted to pH=10 by means of 1.5 g 5% sodium hydroxide. Heat is applied until reaching return temperature, and 40.8 g 50% hydrogen peroxide is fed in within 30 minutes and allowed to continue reacting for another 5 minutes. The reaction mixture is then added to a solution of 25 g sodium sulfite in 72 g water at 60° C. and stirred for 3 hours at 60° C. to achieve a complete peroxide degradation. The product is filtered off the sedimented precipitate and washed with tert.-butyl methyl ether. Methanol and tert.-butyl methyl ether are distilled off the filtrate obtained. Phase separation is performed with the remaining 2-phase residue, and the aqueous phase is extracted twice, each time with 20 g tert.-butyl methyl ether. The combined organic phases are concentrated by means of a rotation evaporator, and the remaining residue is distilled in a vacuum. 13.1 g of distillate are obtained.

### Example 6 (Continuous Process)

13.6 g D-limonene, 61 g methanol and 1.1 g sodium molybdate dihydrate, dissolved in 2 g water, are placed into a double-jacketed reaction tube having a volume of 150 ml, and 0.75 g of 5% sodium hydroxide is added. The reaction mixture is heated to 60° C., and in the course of 30 minutes 20.4 g of 50% hydrogen peroxide are fed into the lower portion of the reactor. Subsequently, 61.2 g 50% hydrogen peroxide and a stirred mixture, consisting of 40.8 g D-limonene, 183 g methanol, 3.3 g sodium molybdate dihydrate, 18 g water and 2.3 g 5% sodium hydroxide are fed in parallel into the bottom portion of the reactor within 45 minutes by means of dosing pumps. The resulting reaction mixture is continuously passed from the upper portion of the reactor into a solution of 50 g sodium sulfite and 144 g water and stirred for 3 hours at 60° C. Further processing is carried out analogous to example 5. 29.1 g of distillate are obtained.

TABLE 1
Contents in the reaction mixture following the reducing step

| | GC area -% | | | | |
|---|---|---|---|---|---|
| | | cis/trans p-2,8- | cis/trans p-1(7),8- | | Sum of |
| | D- | Menthadien-1-ol | Menthadien-2-ol | cis/trans Carveol | products |
| Example | limonene | (Product A) | (Product B) | (Product C) | A, B and C |
| 1 | 5.8 | 35.6 | 36 | 14.7 | 86.3 |
| 2 | 2.9 | 31.9 | 39 | 19.3 | 90.2 |
| 3 | 14.6 | 11.9 | 27.5 | 25.8 | 65.2 |
| 4 | 15 | 18.5 | 36.3 | 24.9 | 79.7 |
| 5 | n.n. | 40.9% | 38.5% | 17.8% | 97.2 |
| 6 | 27.7% | 28.2% | 29% | 11.5% | 68.7 |

Analysis of the Cis/Trans Content of p-2,8-Menthadien-1-ol

The content of cis/trans isomers is determined by means of gas chromatography. The ratio of cis-p-2,8-Menthadien-1-ol to trans-p-2,8-Menthadien-1-ol is about 1:3, and thus approximately corresponds to the ratio indicated in Schenk et. al, Liebigs Ann. Chemie, 674 (1964), 93-117. The ratios of cis- to trans-p-1(7),8-Menthadien-2-ol and of cis- to trans-Carveol also correspond to the ratios indicated in Schenk et al. Therefore, the isomer ratios are the same as in the photo-oxidation process.

1.-15. (canceled)

16. A process for the oxidation of limonene, comprising the reaction of limonene with hydrogen peroxide in the presence of a catalyst containing atoms and/or ions of at least one metal, selected from the group consisting of molybdenum, tungsten, scandium, vanadium, titanium, lanthanum, zirconium, praseodymium, neodymium, samarium, europium, terbium, dysprosium, erbium or ytterbium, characterised in that the molecular weight of the catalyst is less than 2,000 g/mol and that the reaction is performed at a pH value of more than 7.5.

17. The process of claim 16, characterised in that the catalyst contains atoms and/or ions of at least one metal, selected from the group consisting of molybdenum, tungsten, scandium, vanadium, titanium and lanthanum, and preferably of molybdenum and tungsten.

18. The process of claim 16, wherein the catalyst is selected from the group consisting of sodium molybdate, sodium molybdate dihydrate, sodium tungstate, sodium tungstate dihydrate and lanthanum nitrate.

19. The process of claim 16, wherein the reaction is performed in at least one organic solvent.

20. The process of claim 19, wherein the solvents are selected from the group consisting of C1-C8 alcohols and amides.

21. The process of claim 16, wherein the pH value is more than 8.

22. The process of claim 16, wherein the temperature is from 25 to 90° C.

23. The process of claim 16, wherein the pH value is more than 8 and the temperature is in the range from 40° C. to 90° C.

24. The process of claim 16, wherein the pH value is more than 9 and the temperature is in the range from 40° C. to 90° C.

25. The process of claim 16, wherein the amount of catalyst used is 1 to 50 mole percent, based on limonene.

26. The process of claim 16, wherein 2 to 10 molar equivalents of hydrogen peroxide per 1 mole limonene are used.

27. A process for the production of hydroxy derivatives of limonene, comprising the following steps:
(a) oxidation of limonene according to any one of the preceding claims, and
(b) reaction of the mixture obtained in step (a) with a reducing agent.

28. The process of claim 27, performed at a temperature of 25 to 90° C. at a pH value of more than 7.5.
